(19) United States
(12) Patent Application Publication
(10) Pub. No.: US 2023/0032021 A1
(43) Pub. Date: Feb. 2, 2023

(54) ULTRAFINE NEEDLE ENDOSCOPE APPARATUS FOR DEEP INTERSTITIAL EXAMINATION BY WHITE LIGHT IMAGING, AUTOFLUORESCENCE IMAGING AND RAMAN SPECTROSCOPY

(71) Applicants: University Health Network (UHN), Toronto (CA); Ryerson University, Toronto (CA)

(72) Inventors: Alexandre Douplik, North York (CA); Alexandra Easson, Toronto (CA)

(21) Appl. No.: 17/784,522

(22) PCT Filed: Dec. 11, 2020
(86) PCT No.: PCT/CA2020/000135

§ 371 Date: Jun. 10, 2022

Related U.S. Application Data
(60) Provisional application No. 62/946,955, filed on Dec. 11, 2019.

(51) Int. Cl. A61B 10/04; A61B 1/00; A61B 1/07; A61B 1/06; A61B 1/04; A61B 5/00; A61B 1/015; A61B 10/02; A61B 1/12

(52) U.S. Cl. CPC A61B 10/04; A61B 1/00117; A61B 1/07; A61B 1/0669; A61B 1/042; A61B 1/0638; A61B 5/0075; A61B 1/015; A61B 10/0283; A61B 1/126; A61B 1/00154; A61B 1/043; A61B 1/00096; A61B 2010/045

(57) ABSTRACT

Endoscope apparatuses for generating at least one image of a portion of an object are described herein. The endoscope apparatus includes an ultrafine needle adapted for insertion into the object; a fiber probe that is slidably disposed in the ultrafine needle, the fiber probe including a plurality of optical fibers or cores that act as illumination optical fibers and collection optical fibers; an optical assembly that is coupled to the fiber probe, the optical assembly including: at least one transmission optical pathway to provide at least one excitation light signal to the portion of the object to be imaged during use; and at least one return optical pathway that is adapted to transmit reflected light signals from the portion of the object when it is illuminated during use with at least one sensor for generating at least one image via at least one set of optical elements.

### CROSS-REFERENCE TO RELATED APPLICATION

This application claims the benefit of U.S. Provisional Patent Application No. 62/946,955 filed Dec. 11, 2019 and the entire contents of U.S. Provisional Patent Application No. 62/946,955 are hereby incorporated herein in its entirety.

### FIELD

The present disclosure relates to endoscopy and more particularly to needle endoscopy which may be used to perform deep interstitial biopsy imaging guidance, diagnostics and treatment monitoring.

### BACKGROUND

Breast cancer is the most common cancer in Canadian women, and early detection saves lives. Best practice recommendations include advising all women aged 50-74 to have a screening mammogram every 1-2 years. While mammographic screening reduces breast cancer death by 20-48% [1], 8-12% of women are called back for a false positive result for more imaging [2,3,4]. If on repeat imaging the lesion is deemed indeterminate or suspicious, 1.5% require an invasive core tissue biopsy or surgery, resulting in significant patient anxiety, pain and complications, and use of health care resources. In 2011, the Ontario Breast Screening Program led to 4,582 biopsies, and 2,588 (56%) of these biopsies were non-cancerous.

Although techniques for image-guided biopsies continue to improve, no current technique can: (a) reliably target the correct area (leading to repeat biopsies or additional tests), (b) avoid biopsy in questionable lesions, or (c) alter the fact that biopsies are invasive (e.g. bleeding, bruising), expensive and painful. Moreover, because no current technique looks directly at the tissue to ensure that the correct area is sampled, repeat biopsies may be needed if pathology and imaging does not correlate and several areas may need biopsy in the same patient. Also, it takes a while to obtain the biopsy results which can be psychologically distressing for patients, as several days pass between steps, the entire process can take weeks, and most results will not be cancer. Clearly, reliable, less invasive methods of diagnosis for breast cancer and other conditions (e.g. prostate or thyroid cancer) would have a significant impact on patients.

Remarkably, there is a dearth of literature around the patient experience throughout this process considering how many women are affected. Population-based studies report 50-61% of women between 50-74 years of age will be called back in 10 years of screening [2]. In Alberta, 213,867 women had screening mammograms from 2006-2008, with a false positive rate of 9.4% (22,751 women) and an 8.1 benign biopsy rate per 1,000 women screened [3]. A systematic review [4] concluded some patients report significant psychological distress from a false positive mammogram, and that it can take up to 3 years for this distress to abate. These authors also found that these women were less likely to return for their next scheduled screening mammogram after a call back (Relative Risk (RR)=1.2), and importantly, that this tendency was increased if they received a fine-needle biopsy (RR=1.8) or a more invasive biopsy (RR=2.29).

There is also remarkably little reported about how the size and number of biopsies affect the patient. Standard core biopsies used routinely at the discretion of the interventional radiologists for tissue diagnosis vary by size (7-14 Gauge=4.5-1.63 mm diameter), the number of samples taken (usually >3), and whether vacuum is used. Smaller diameter biopsies cause less tissue destruction, and also less pain and bleeding but at the expense of diagnostic yield [8].

### SUMMARY OF VARIOUS EMBODIMENTS

According to one broad aspect of the teachings herein, there is provided an endoscope apparatus for obtaining light signals for generating at least one image of a portion of an object using at least one imaging modality, wherein the apparatus comprises: an ultrafine needle having a body with first and second ends, the first end being adapted for insertion into the object; a fiber probe that is slidably disposed in the ultrafine needle; an optical cable having a body with first and second ends, the body having an outer cladding that surrounds a plurality of illumination optical fibers and a plurality of collection optical fibers, the first end of the optical cable forming the fibre probe; a port having first and second ends, the first end of the port being coupled to the second end of the optical cable and the second end of the port being connected to at least one light source and at least one sensor where the at least one light source is adapted to provide at least one excitation light signal to the portion of the object to be imaged during use and at least one sensor is adapted to receive reflected light signals from the portion of the object when it is illuminated during use for generating the at least one image via.

According to one broad aspect of the teachings herein, there is provided an endoscope for obtaining light signals for generating multiple images of a portion of an object using several imaging modalities, wherein the endoscope comprises: an ultrafine needle having a body with first and second ends, the first end being adapted for insertion into the object; a fiber probe that is slidably disposed in the ultrafine needle, the fiber probe including a plurality of optical fibers or cores that act as illumination optical fibers and collection optical fibers; an optical assembly that is coupled to the fiber probe, the optical assembly including: at least one transmission optical pathway that is adapted to optically couple at least one of the optical fibers or cores with at least one light source via at least one set of optical elements to provide at least one excitation light signal to the portion of the object to be imaged during use; and at least one return optical pathway that is adapted to optically couple at least one of the optical fibers or cores that receive reflected light signals from the portion of the object when it is illuminated during use with at least one sensor for generating at least one image via at least one set of optical elements.

According to one broad aspect of the teachings herein, there is provided an endoscope for obtaining light signals for generating multiple images of a portion of an object using several imaging modalities, wherein the endoscope comprises: an ultrafine needle having a body with first and second ends, the first end being adapted for insertion into the object; a fiber probe that is slidably disposed in the ultrafine needle, the fiber probe including a plurality of optical fibers or cores that act as illumination optical fibers and collection optical fibers; an optical assembly that is coupled to the fiber probe, the optical assembly including: at least two transmission optical pathways that are adapted to optically couple at least one of the optical fibers or cores with at least two light sources via at least two sets of optical elements to provide at least two excitation light signals to the portion of the object to be imaged during use; and at least two return optical pathways that are adapted to optically couple at least one of the optical fibers or cores, that receive reflected light signals from the portion of the object when it is illuminated during use, with at least two sensors for generating at least two images via at least two second sets of optical elements, wherein the at least two excitation light signals and the at least two sensors are adapted for generating two or more of a white light image, an Autofluorescence image and a Raman image.

In at least one embodiment, the body of the ultrafine needle has an outer diameter of about 200 microns.

In at least one embodiment, the body of the ultrafine needle has an inner diameter of about 120 microns.

In at least one embodiment, the length of the ultrafine needle is about at least 3 to 4.5 cm or longer.

In at least one embodiment, the fiber probe has optical fibers or cores adapted to provide a resolution from about 250 to 6,000 pixels.

In at least one embodiment, the optical assembly comprises: a first transmission optical pathway that is adapted for sending a broadband excitation light signal from a first light source that is a broadband light source via a first set of optical elements to the objective for transmission to the portion of the object being imaged; and a first return optical pathway that is adapted for sending first reflected signals from the portion of the object being imaged in response to the broadband excitation light signal from the objective to a camera sensor for white light color imaging and to a spectral imaging sensor for spectral imaging.

In at least one embodiment, the optical assembly comprises: a second transmission optical pathway for sending a second excitation light signal from a second light source that provides a 785 nm excitation light signal via a second set of optical elements to the objective for transmission to the portion of the object being imaged; and a second return optical pathway for sending second reflected signals from the portion of the object being imaged in response to the second excitation light signal from the objective to a light sensor for obtaining Raman images for wavelengths at about 785 nm.

In at least one embodiment, the optical assembly comprises: a third transmission optical pathway for sending a third excitation light signal from a third light source that provides a 532 nm excitation light signal via a third set of optical elements to the objective for transmission to the portion of the object being imaged; and a third return optical pathway for sending third reflected signals from the portion of the object being imaged in response to the third excitation light signal from the objective to a third light sensor for obtaining Fluorescence images or Raman and Fluorescence images for wavelengths less than about 765 nm.

In at least one embodiment, the optical assembly further comprises a notch filter for eliminating cross-talk between different optical pathways during use.

In at least one embodiment, the apparatus includes a second channel that is adapted to be coupled to a suction device for collecting biopsy tissues and cells during use from the portion of the object being imaged.

In at least one embodiment, the apparatus includes a third channel that is adapted to be coupled to a rinsing device to provide a rinsing solution during use to the portion of the object being imaged.

In at least one embodiment, The apparatus of any one of claims 1 to 13, wherein the ultrafine needle is made of stainless steel.

In another aspect, in accordance with the teachings herein, there is provided a method for generating at least one image of a portion of an object using an endoscope apparatus as defined in any one of claims 1 to 14, wherein the method comprises: inserting the ultrafine needle into the object; coupling a broadband light source to the fiber probe; generating at least one excitation light signal using at least one light source; receiving at least one reflected light signal from the portion of the object; and transmitting the at least one reflected light signal to at least one sensor for generating a white light image, an autofluorescence image and/or a Raman spectral image, wherein the ultrafine needle contains the fiber probe.

In at least one embodiment, inserting the ultrafine needle into the object comprises: inserting a wire into the ultrafine needle; inserting the wire and the ultrafine needle into the object; removing the wire from the ultrafine needle; and inserting the fiber probe into the needle.

In at least one embodiment, the method comprises creating an interstitial channel for inserting the wire and the first end of the ultrafine needle into the object.

In at least one embodiment, the object is ex vivo tissue or in vivo tissue.

In at least one embodiment, the method comprises generating a thyroid image, a prostate image, a breast image or an ascites image.

In at least one embodiment, the method further comprises coupling the endoscope apparatus to a rinsing device and providing a rinsing solution to the object prior to performing imaging.

In at least one embodiment, the method further comprises coupling the endoscope apparatus to a suction device and obtaining a biopsy sample from the object.

In at least one embodiment, the method further comprises obtaining a thyroid biopsy, a prostate biopsy, or an ascites biopsy.

In another aspect, in accordance with the teachings herein, there is provided a use of an endoscope apparatus for obtaining a thyroid biopsy, a prostate biopsy, or an ascites biopsy where the endoscope apparatus is defined according to at least one of the embodiments described herein.

In another aspect, in accordance with the teachings herein, there is provided use of an endoscope apparatus for obtaining an image of a portion of an object, where the endoscope apparatus is defined according to any of the embodiments described herein.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A is a schematic of an example embodiment of an ultrafine needle endoscope apparatus with a detection head in accordance with the teachings herein.

FIG. 1B is a schematic of an example embodiment of a detection head with an optical assembly that can be used to interface the ultrafine needle endoscope of FIG. 1A with various light sources and light sensors.

FIG. 2A is an example embodiment of a prototype ultrafine needle endoscope in accordance with the teachings herein.

FIG. 2B is a view of an ultrafine needle that can be used with the ultrafine needle endoscope of FIG. 2A.

FIG. 2C is an end view of an optical cable that can be used with the ultrafine needle endoscope of FIG. 2A.

FIG. 2D shows an example of a circular arrangement of cores for an Outer Diameter (OD) of 150-microns, a Core Diameter (CD) of 1 micron, and a spacing of 1 micron.

FIG. 2E shows an example of a rectangular arrangement of cores for an OD of 150-microns, a CD of 1 micron, and a spacing of 1 micron.

FIG. 2F shows an example of a circular arrangement of cores for an OD of 150-micron, CD of 3 micron, and spacing of 1 micron.

FIG. 2G shows an example of a rectangular arrangement of cores for an OD of 150-micron, CD of 3 micron, and spacing of 1 micron.

FIG. 3 is a view of another example embodiment of an ultrafine needle endoscope with additional attachments for providing increased functionality in accordance with the teachings herein.

FIGS. 4A-4B are white light color images that have been obtained using a prototype ultrafine needle endoscope apparatus.

FIGS. 4C-4D are autofluorescence images that have been obtained using the prototype ultrafine needle endoscope apparatus used to obtain the images in FIGS. 4A-4B.

FIG. 5A shows results for autofluorescence images obtained from ex vivo breast tissue samples on several patients using a prototype ultrafine needle endoscope apparatus having a 550 micron outer diameter.

FIG. 5B shows results for Raman spectroscopy performed on ex-vivo normal breast tissue from a first patient using a prototype ultrafine needle endoscope apparatus having a 550 micron outer diameter.

FIG. 5C shows results for Raman spectroscopy performed on ex-vivo breast tissue having a tumor from a second patient using a prototype ultrafine needle endoscope apparatus having a 550 micron outer diameter.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide examples of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the example embodiments described herein.

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate that two elements or devices can be directly connected to one another or connected to one another through one or more intermediate elements or devices via an electrical or optical signal, an electrical or optical connection, an electrical element, an optical element or a mechanical element depending on the particular context. Furthermore, certain coupled electrical elements may send and/or receive data.

Unless the context requires otherwise, throughout the specification and claims which follow, the word “comprise” and variations thereof, such as, “comprises” and “comprising” are to be construed in an open, inclusive sense, that is, as “including, but not limited to”.

It should also be noted that, as used herein, the wording “and/or” is intended to represent an inclusive-or. That is, the expression “X and/or Y” is intended to mean X or Y or both, for example. As a further example, the expression “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Imaging by optical means is one of the diagnostic options when examination can be facilitated at high resolution (about 1-10 microns). Such a high resolution can be critical when searching for small lesions and early pathological states. Fluorescence imaging can provide strong functional and structural content variation but requires external dyes/agents. Autofluorescence (AF) imaging does not require dyes and provides clinical diagnostic accuracies in the range of 75-85% specifically when combined with diffuse reflectance imaging. Also an external clinically approved dye can be used, such as ICG or fluorescein. Raman spectroscopy can provide the highest differentiation as it can provide high structural and functional variation between cancerous and normal tissue types, with a sensitivity/specificity of 85-92%/88-100% in breast, 97%/98% in brain and 100%/98% in gastrointestinal cancer [11]. Raman spectroscopy appears to be particularly useful in its diagnostic ability to distinguish between early cancerous changes. Given the wide spectrum of premalignant, in situ, and malignant changes that are found in the breast, this ability may be particularly valuable. However, signal acquisition times are longer and interference from AF and other signals can prove limiting. Advantageously, the inventors have realized that the combination of the three modalities of white light, AF and Raman spectroscopy into one optical diagnostic instrument may provide an optimum combination for fast and effective diagnosis.

The main limitation of diagnostic optical imaging is light penetration depth for biological tissue as it is currently limited to 1-3 mm depending on the color of light used (i.e. the wavelength range). This limit is prescribed by tissue optical properties, namely by high light scattering due to cell membranes, subcellular components, and interfaces between different tissue types, as well as high light absorption by intrinsic chromophores such as melanin and hemoglobin. Hence, most clinical applications of optical imaging are currently limited to superficial or shallow examination of epithelium and mucosa, and comprise either handheld, free space, microscopy like devices, or endoscopy devices. This is exacerbated in the case of diagnostic breast, thyroid or prostrate imaging, the lesions in question are at depths from the skin surface of up to 3-5 cm or more, in a minority of cases for patients having big anatomical sizes, which is not currently reachable with the optical resolution that is achievable using the methods mentioned above.

However, while not all pathological loci can be reached through natural ducts such as the Gastrointestinal tract (GI), lungs or even small ducts like breast ducts, an endoscope apparatus that uses an ultrafine needle, in accordance with the teachings herein, can be used to penetrate through tissue in order to perform small invasive or interstitial endoscopy. For example, an ultrafine needle with an outer diameter that is less than about 200 microns in size can be squeezed between capillaries and nerves.

In another aspect of the teachings herein, an optics combination or multimodal imaging approach may be used to optimize the clinical diagnostic accuracy for the endoscope apparatus. For example, white light images can confirm location, AF images have a diagnostic accuracy of about 70-80%, and if needed, Raman spectroscopy/imaging (with an accuracy 98-99%) can be added to maximize the diagnostic accuracy. Accordingly, a combination of these imaging modalities will increase clinical diagnosis accuracy.

For example, the inventors have found that an image produced from the end of an endoscope (0.7 mm in outer diameter, 3,000 pixels) attached to an AF imaging system can distinguish between cancerous and non-cancerous breast tissue when placed into a breast duct [9]. The inventors have also developed a system using a second generation AF-coupled endoscope (diameter 0.55 mm, 6,000 pixels) that successfully distinguished between normal and cancerous breast tissue when the endoscope was placed directly into breast tissue through a standard 14 Gauge biopsy trocar to look at the lesion of interest.

Accordingly, in accordance with the teachings herein, there is provided an endoscope (i.e. a microendoscope) apparatus with multispectral/multimodal optical imaging capability for deep interrogation of organ tissues to obtain optical data from deep body structures at high resolution. The multimodal imaging includes in color imaging, fluorescence and/or Raman modalities while being minimally invasive and allowing for direct real time diagnostic optical imaging of breast tissue to depths of 4 cm during screening to improve the patient diagnostic cancer experience in the outpatient clinic. The endoscope of the present teachings comprises an acupuncture size hollow ultrafine needle along with light guiding fibers to facilitate the collection of reflected light for performing endoscopy and generating at least one of: (a) white light color (W) images, (b) Autofluorescence (A) images and (c) Raman (R) images (also known as WAR), which may be used to image cancer or other types of pathologies. Since the ultrafine needle that is used is very small in diameter this allows for small invasive interstitial endoscopy in which there is little or no pain, little of no bleeding, and the tissue/cells will self-repair themselves. Therefore, larger holes are not critical for access from the surface of a patient's skin when the endoscopy is performed in vivo based on an using an ultrafine needle such as those described herein. It should be noted that the ultrafine needle may also be referred to as a microneedle.

In at least one embodiment described herein, a microendoscope can be used which provides for a high imaging resolution (e.g. 1-3 microns) at a depth of 4-4.5 cm due to the small diameter of the needle and fibre probe of the endoscope. Such an imaging apparatus has not previously been implemented. For example, previously, a conventional high resolution imaging technique based on Optoacoustic Tomography can only provide a resolution of not more than 30-40 microns at a depth of not more than 3 cm and this technology is also more cumbersome to use as it requires the object being imaged to be immersed into water which acts as a sound coupling media. However, the endoscopes apparatuses described herein do not require any external coupling media, can be used in-vivo and have a resolution that is one order of magnitude more sensitive. Combining this resolution/depth superiority with diffuse reflectance, fluorescence and/or Raman imaging modalities may greatly improve cancer diagnostics.

In some cases, a hollow needle or trocar can be placed directly into the tissue at the time of biopsy and be coupled to an endoscope that can be used to collect accurate diagnostic imaging data by penetrating deeper into a patient's body to look directly at the tissue in question and measure its diagnostic optical properties, and may also be used to diagnose malignancy without the need for pathologic tissue confirmation. Accordingly, the endoscope apparatuses described herein may be used to obtain diagnostic information from deep body structures at high resolution.

Examples of medical applications in which endoscope apparatuses of the present teachings may be used include at least one of imaging, diagnostics and biopsy of a patient's skin, breast, thyroid, prostate or ascites (e.g. fluid in the peritoneal cavity).

In another aspect, use of an endoscope apparatus in accordance with the teachings herein may allow for at least one of: (1) faster healing, (2) improved real time tissue diagnosis (i.e. a small invasive examination at the time of consultation and/or early diagnostics), (3) provision of visual guidance during a biopsy procedure, (4) a reduced need for biopsy in indeterminate lesions based on visual information obtained using the endoscope, (5) confirmation of lesion location, or (6) even avoidance of surgery when the tissue being assessed is determined to be benign based on additional information provided by the endoscope.

In at least one embodiment, an endoscope apparatus capable of being used in accordance with the teachings herein includes an ultrafine needle having an outer diameter on the order of about 200 microns.

In contrast, the smallest commercially available high resolution imaging endoscopes contain 3000 pixels/fibers and have an outer diameter that is 460 microns [12]. At the smallest extreme, acupuncture uses needles with outer diameters less than 200 microns (e.g. 34 Gauge (G) and smaller), inserting these needles to a depth up to 3-5 cm for up to 60 min [13] without excessive bleeding or pain [14]. It can be assumed that the damage created by these needles is self-repairable or non-critical when approached from the skin.

However, it may be possible that needles with outer diameter less than the distances between blood capillaries (200 microns) may not destruct or collapse the blood supply infrastructure, but rather move tissue elements apart without pain or other consequences. One study found a significant difference in complications and pain between syringes measuring 32-33 G (230-210 micron external diameter) and 34 G (smaller than 200 microns), calling the 34 G syringe (185-190 micron external diameter) a “non-painful” threshold [15].

Accordingly, a diagnostic optical endoscope that is 34 G or smaller, in accordance with the teachings herein, may have a significant effect in cancer screening and biopsy guidance, by allowing a small invasive examination at the time of biopsy, significantly reducing tissue destruction, complications and potentially the need for biopsy. In particular, several devices as small as 34 G may be inserted at different directions to ensure that a lesion is adequately targeted, to facilitate a diagnosis at the time of consultation which would likely not cause pain, not require anesthesia, or leave bruising or cellular changes in the tissue.

If used in a national screening program, the minimally invasive technology described herein may provide radiologists with a reliable tool to guide the biopsy needle to ensure that the appropriate tissue is sampled and avoid the need for biopsy if benign, which may result in significant clinical impact by minimizing tissue destruction, and improving the diagnostic screening experience.

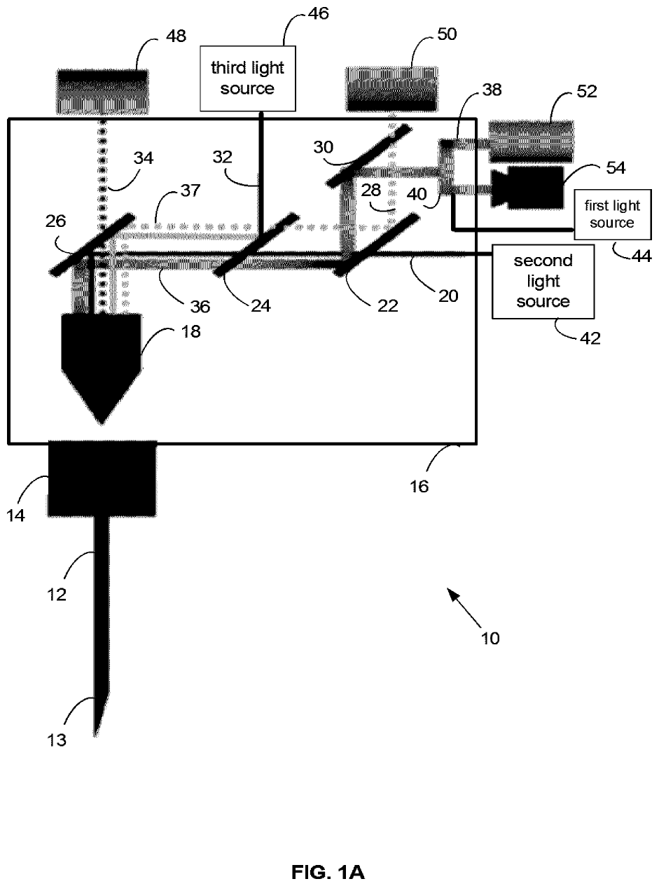

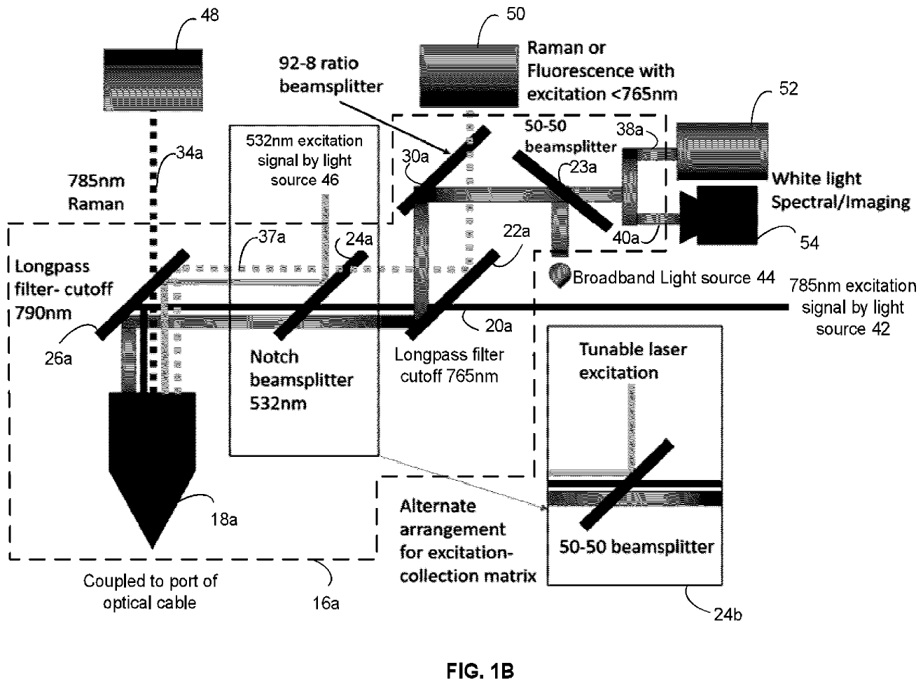

Referring now to FIG. 1A, shown therein is a schematic of an example embodiment of an endoscope apparatus 10 (also known as an ultrafine needle endoscope apparatus or a microendoscope apparatus) including an ultrafine needle endoscope in accordance with the teachings herein. The endoscope apparatus 10 comprises an ultrafine needle 13, a fibre probe 12, a coupling adaptor 14, and a detection head 16. The ultrafine needle 13 has a distal end for insertion into a portion of an object to be imaged and a proximal end that is receives the fibre probe 12 which is connected to the coupling adaptor 14. A second end of the coupling adaptor 14 is coupled to an optical assembly via an objective 18 of the detection head 16. The adaptor 14 is used to maintain a fixed geometric relationship with the optical fibers of the fiber probe 12 with the objective 18. The objective 18 may be implemented using at least two optical lenses. The ultrafine needle endoscope apparatus 10 can be used to provide white light, fluorescence and/or Raman sensing, depending on the implementation of the optical assembly and associated light sources and sensors.

The fiber probe 12 is slidably received within the ultrafine needle 13 and can be removed and reinserted as needed during use. The fiber probe 12 can be quite long in length and flexible except for the portion of the fiber probe 12 at the adaptor 14 where the fibers in the fiber probe 12 are kept in a fixed geometric relation with the objective 18. Depending on its configuration the fiber probe 12 can be adapted so that the apparatus 10 can be used for imaging, for spectroscopy or for both imaging and spectroscopy. For example, the fibre probe 12 may be implemented as an imaging fiber probe that can include many cores such as about 7 to 20 or more. Alternatively, the fiber probe 12 may be implemented using a spectroscopy data collection fiber probe that has less than 20 cores. The term “core” means a single fiber if the imaging probe is a fiber bundle or a single core if the fiber probe is a multicore fibre or a fibre plate.

The objective 18 of the optical assembly is optically coupled to the adapter 14. The optical assembly includes at least one first transmission optical pathway that is coupled to the objective 18 which is then optically coupled to a plurality of optical fibers within the fiber probe 12. The optical fibers in the fiber probe 12 act as both illumination optical fibers and collection optical fibers based on the optical elements and optical geometry of the optical assembly and the light sources and sensors to which the optical assembly is coupled. In particular, the optical assembly is coupled at least one light source, via at least one first set of optical elements, to provide at least one excitation light signal to the portion of the object to be imaged during use. The optical assembly also includes at least one return optical pathway that is coupled to the objective 18 and is adapted to optically couple a plurality of optical fibers, that act as collection fibers when they receive reflected light signals from the portion of the object when it is illuminated during use, with at least one sensor via at least one second set of optical elements. The at least one sensor generates at least one image of the portion of the object being illuminated based on the reflected light signals that are being provided to it.

The detection head 16 includes a housing that is coupled to at least one light source 44 and houses the optical assembly that has several optical pathways and optical elements that are used for sending light signals to or receiving light signals from the portion of the object being imaged using one or more types of imaging. For instance, in the example embodiment of FIG. 1A, the optical assembly includes optical pathways 36, 38, 40, 20, 28, 32 and 34, at least one beamsplitter 30, at least one optical filter 26 and 22, and at least one optical notch beamsplitter 24 for providing an excitation light signal from at least one of the excitation light sources 42, 44 and 46 to the objective 18 for transmission to the ultrafine needle 12 as well as for receiving at least one reflected signal and providing them to a corresponding sensor such as a Raman sensor 48, a Raman and/or Fluorescence sensor 50, a white light color sensor 52 and a spectral sensor 54. In some embodiments, the white light color sensor 52 and the spectral sensor 54 may be implemented using a spectral imaging device.

In various embodiments, some of the optical pathways, optical elements, light sources and light sensors may be optional such that the endoscope apparatus 10 is used for generating any sub-combination of (a) white light color images, (b) spectral images, (c) Raman images and (d) autofluorescence images.

Referring now to FIG. 1B, shown therein is a schematic of an example embodiment of a detection head with an optical assembly that can be used to interface the ultrafine needle endoscope 13 of FIG. 1A with various light sources and light sensors. It should be noted that the optical assembly shown in FIG. 1B is just one example and there can be other optical assembly embodiments with a different number and/or arrangement of optical elements for performing the same functions.

The optical assembly includes a first transmission optical pathway 36 (see FIG. 1A) for sending a broadband excitation light signal from the light source 44, which is a broadband light source, via optical elements 23a, 30a, 22a, 24a and 26a to the objective 18 for transmission to the portion of the object being imaged. First reflected signals from the portion of the object being imaged in response to the broadband excitation light signal travel from the objective 18 back along a first return optical pathway including the first optical pathway 36 to beam splitter 23a which separates the first reflected signals to cause them to follow optical pathways 38a and 40a to a camera sensor 52 for white light color imaging and to a spectral imaging sensor 54 for spectral imaging. In this example embodiment, the optical element 23a is a 50-50 beamsplitter, the optical element 30a is a 92-8 ratio beamsplitter, the optical element 22a is a longpass filter with a cutoff of 765 nm, the optical element 24a is a 532 nm notch beamsplitter, and the optical element 26a is a longpass filter with a cutoff of 790 nm. In some embodiments, a tunable laser may be used as the light source 46 and a 50-50 beamsplitter may be used instead of the 532 nm notch beamsplitter.

The optical assembly also includes a second transmission optical pathway for sending a second excitation light signal from the light source 42, which provides a 785 nm excitation light signal, via the optical elements 20a, 24a and 26a to the objective 18 for transmission to the portion of the object being imaged. Second reflected signals from the portion of the object being imaged in response to the second excitation light signal travel from the objective 18 along second return optical pathway 34a via optical element 26a to the light sensor 48 which can be used to obtain a Raman images for wavelengths at about 785 nm.

The optical assembly also includes a third transmission optical pathway for sending a third excitation light signal from the light source 46, which provides a 532 nm excitation light signal, via the optical elements 24a and 26a to the objective 18 for transmission to the portion of the object being imaged. Third reflected signals from the portion of the object being imaged in response to the third 37a via optical elements 26a, 24a, 22a and 30a to the light sensor 50 which can be used to obtain Fluorescence or Raman and Fluorescence images for wavelengths less than about 765 nm.

It should be noted that there may be an alternative embodiment in which only one of: (a) the first transmission and first return optical pathways; (b) the second transmission and second return optical pathways; and (c) the third transmission and third return optical pathways along with the corresponding optical elements are included in the optical assembly in which case only one of the types of images that correspond to the transmission and return pathways included in the optical assembly can be generated.

It should also be noted that there may be an alternative embodiment in which only two of: (a) the first transmission and first return optical pathways; (b) the second transmission and second return optical pathways; and (c) the third transmission and third return optical pathways along with the corresponding optical elements are included in the optical assembly in which case only two of the types of images that correspond to the transmission and return pathways included in the optical assembly can be generated.

It should also be noted that in some embodiments a camera or a spectrometer may be used for at least one of the image sensors 48, 50, 52 and 54. Alternatively, in some embodiments other optical elements, including an optical slit and a diffraction grating, may be used for at least one of the sensors 48, 50, 52 and 54.

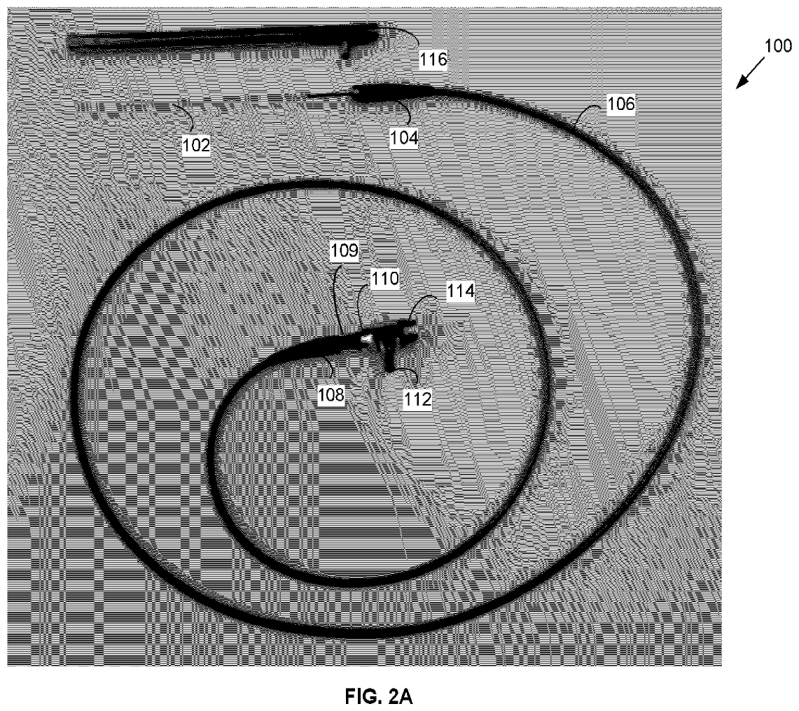

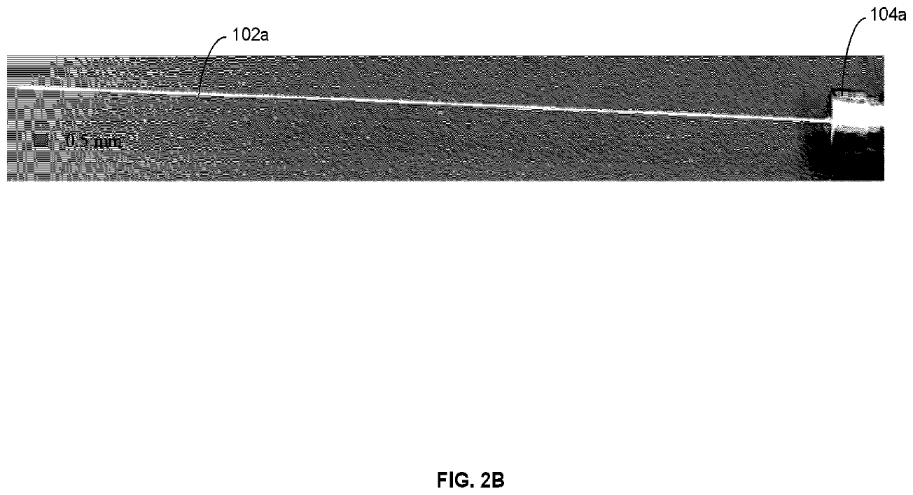

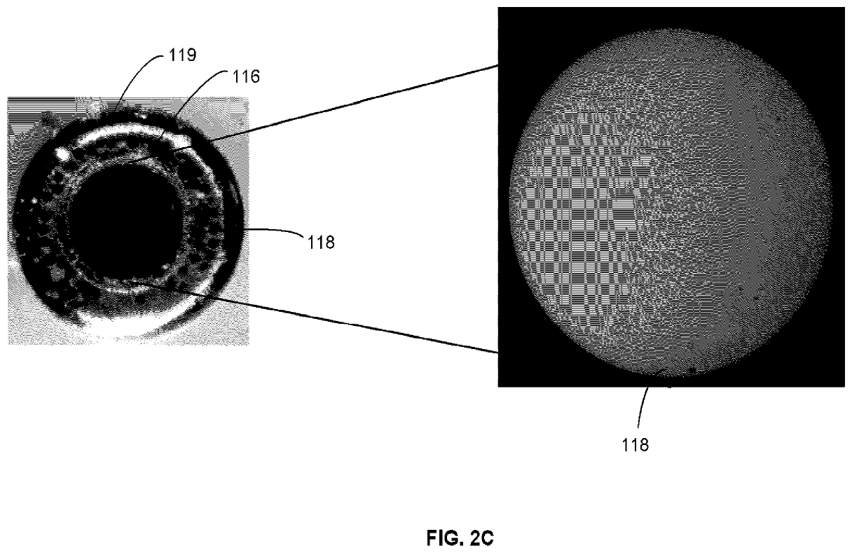

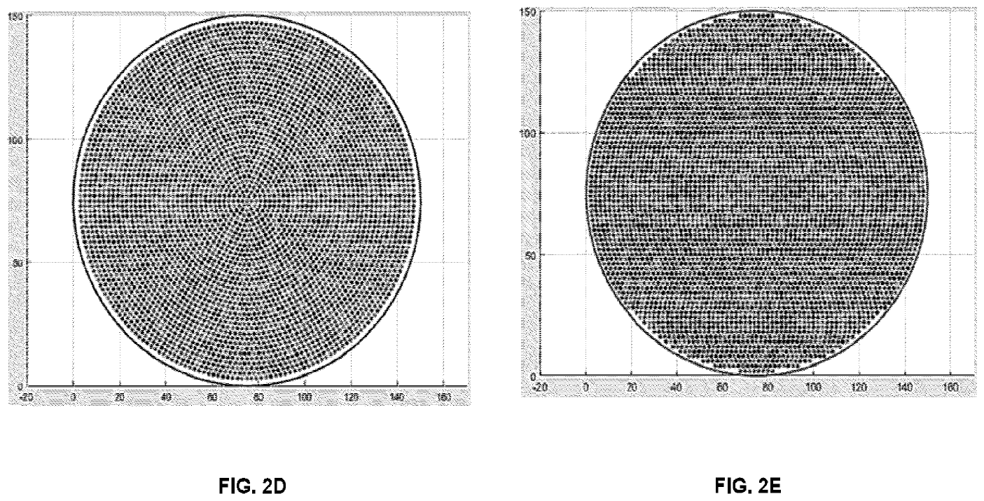

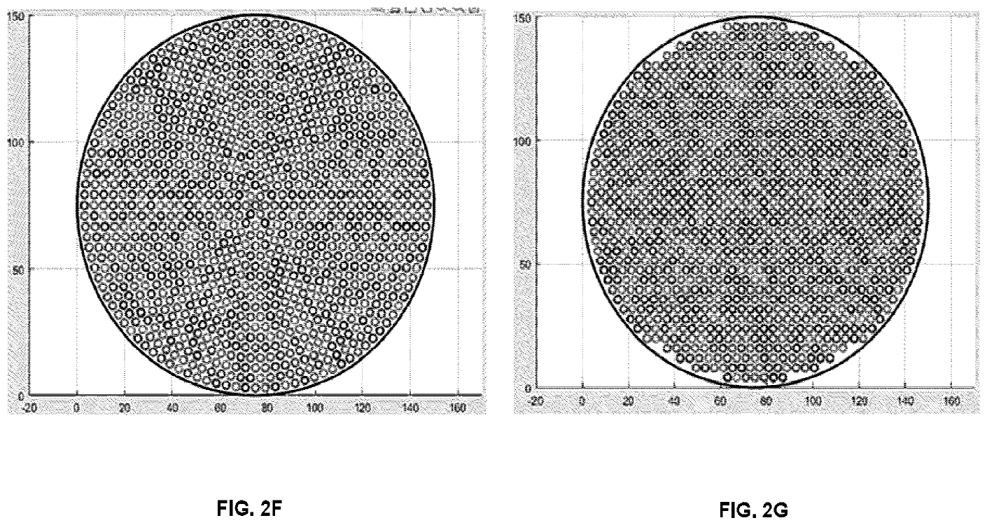

Referring now to FIG. 2A, shown therein is an example embodiment of a prototype ultrafine needle endoscope 100 in accordance with the teachings herein. The needle itself is not shown but the endoscope 100 includes a fiber probe 102, an optical cable 106, a port 109, an optical coupler 110 and a removable storage cylinder 116. The removable storage cylinder 116 is optional and can be used for protecting the fiber probe 102.

The fiber probe 102 has a first distal end that is inserted into a proximal end of an ultrafine needle that has a distal end that is tapered for insertion into an object to image a portion of the object. The optical cable 106 has a body with first and second ends 104 and 108. The optical cable 106 has an outer cladding that surrounds a plurality of optical fibers where some of the optical fibers are used as illumination optical fibers and some of the optical fibers are used as collection optical fibers (e.g. see FIG. 2C). The first end 104 is shaped to removably receive the container 116. The port 109 has first and second ends with the first end being coupled to the second end 108 of the optical cable 106 and the second end of the port 109 having a first channel.

The optical coupler 110 has a first end that is optically coupled to the first channel of the port 109, a first coupling channel 112 that is adapted to optically couple the plurality of illumination optical fibers with at least one light source to provide an excitation light signal to the portion of the object to be imaged during use. The optical coupler 110 also has a second coupling channel 114 that is adapted to optically couple with the plurality of collection fibers to receive reflected light signals from the portion of the object when it is illuminated during use. The second coupling channel 114 may then be coupled to a sensor. The optical coupler 110 includes a beam splitter. In this example embodiment, the first coupling channel 112 is a 90 degrees port and the second coupling channel 114 is a straight port while the ultrafine needle has an outer diameter of about 120 microns and a length of about 45 mm.

Referring now to FIG. 2B, shown therein is a view of an example embodiment of an ultrafine needle 102a that can be used with the ultrafine needle endoscope of FIG. 2A or it may be adapted for use with the fiber probe 13 of FIG. 1A. The ultrafine needle 102a includes a second end 104a that may be coupled to the optical cable 106.

In at least one embodiment, the ultrafine needle 102a may have a length in the range of about 35 to 45 mm or longer.

In at least one embodiment, the ultrafine needle 102a may have an outer diameter of about 200 microns or smaller (which is comparable to an acupuncture needle). For example, the ultrafine needle may be a 34 gauge (G) or smaller needle.

For example, the ultrafine needle 102a may be about 195 microns in outer diameter and a length of about 45 mm which may be used for breast, thyroid, prostrate and ascites imaging.

In at least one embodiment, the ultrafine needle 102a may have an inner diameter that may be about 120 microns or smaller.

In at least one embodiment, the ultrafine needle 102a may be made from medical grade stainless steel.

In at least one embodiment, the ultrafine needle 102a may be a coring needle with an outer diameter of about 200 microns or less and an inner diameter of about 100 microns or less, a length of about 45 mm or more, and sharpened to a beveled point at an angle of 12 degrees plus or minus 5 degrees. Such a needle may be fabricated of medical grade stainless steel, for example, by use of various techniques. In some embodiments, the medical grade stainless steel is rolled from appropriately graded sheet metal of suitable thickness into an initial outer diameter, iteratively reduced in a floating process to the desired outer and inner diameters, welded into fine tube, annealed, passed through a straightening machine, cut to length, and beveled on one end to the desired degree of bevel.

In at least one embodiment, the needle can be inserted into the patient's body or elsewhere with a metal wire kept inside the inner channel of the needle to improve the needle's durability and reduce its bending during insertion, and, once the needle is inserted at the right depth then the wire may be pulled out and the fiber probe is then inserted (i.e. the microneedle with an fiber probe disposed within the channel of the microneedle forms a portion of the endoscope).

Referring now to FIG. 2C, shown therein is an end view of a distal end of an optical cable that can be used with the ultrafine needle endoscope of FIG. 2A. The optical cable comprises two sets of optical fibers where the first set comprises a plurality of optical fibers 116 for illuminating a portion of an object for imaging. The optical fibers 116 can be referred to as illumination fibers or illumination optical fibers that are arranged along the periphery of the optical cable. The second set of optical fibers comprises a plurality of optical fibers 118 to receive reflected light which are reflected by the portion of the object when it is illuminated. The optical fibers 118 can be referred to as collection fibers or collection optical fibers that are arranged along a central longitudinal channel of the optical cable. In this example embodiment, the endoscope has a diameter of about 0.55 mm. Both sets of optical fibers 116 and 118 are encapsulated within a casing 119 for protection and to avoid any leakage of optical signals throughout the length of the optical cable. The casing 119, also known as an outer cladding, can be made from metal or another suitable material.

In certain embodiments, there may be enough optical fibers, depending on the outer diameter of the endoscope, for providing a certain amount of resolution. For example, for an endoscope having an outer diameter of 550 microns, the resolution may be about 6,000 pixels while for an endoscope having an outer diameter of about 80 to 120 microns the resolution may range from about 250 to 3,000 pixels.

The optical fibers 116 can be arranged as a fibre bundle (as shown) or in an alternative embodiment as a flexible fiberoptic plate or multicore fibre that has a dimension allowing for insertion of it into the metal needle. For example, in this alternative embodiment there can be up to 2,000 single fibers or optical core which in turn form imaging pixels via delivery of single or multiple waveguide modes.

For either of the embodiments shown in FIGS. 1A and 2A, there can be variations on how the optical fibers (i.e. cores) are dimensioned, arranged and spaced apart, as well as the size of the OD, which will affect the number of pixels and resolution for some of the images that are generated. A single core provides one imaging pixel. In addition, the size of a core affects the number of light wave modes that can be transmitted using the core. For example a one micron core cannot facilitate the transmission of a color image while a three micron core can transmit enough light wave modes to support a color image. FIG. 2D shows an example of a circular arrangement of cores for an OD of 150-microns, a CD of 1 micron, and a spacing of 1 micron. FIG. 2E shows an example of a rectangular arrangement of cores for an OD of 150-microns, a CD of 1 micron, and a spacing of 1 micron. FIG. 2F shows an example of a circular arrangement of cores for an OD of 150-micron, CD of 3 micron, and spacing of 1 micron. FIG. 2G shows an example of a rectangular arrangement of cores for an OD of 150-micron, CD of 3 micron, and spacing of 1 micron.

Table 1 shows a plurality of different design options for OD ranging from 80 to 250 microns for both circular and rectangular arrangement of cores for a CD of 1 micron. Table 2 shows a plurality of different design options for OD ranging from 90 to 250 microns for both circular and rectangular arrangement of cores for a CD of 3 micron. The space between the cores (and therefore pixels) is preferably be more than a wavelength to avoid cross talk between the adjacent cores (and therefore the adjacent pixels) due to the evanescent radiation; for example a spacing of 0.8-1 microns is recommended. A rectangular arrangement is considered to be more efficient to fit more cores within a certain Outer Diameter (OD), but it is also a more technically challenging to implement.

TABLE 1
Outer diameter (OD) of microfiber vs. number of pixels for two
arrangements with: Core Diameter (CD): 1 micron (Black &
White image only or single mode cores) and edge-edge spacing
between cores of 1 micron

| | Circular Arrangement | Rectangular Arrangement |
|---|---|---|
| OD | No. of Pixels | No. of Pixels |
| 80 | 1141 | 1179 |
| 85 | 1261 | 1352 |
| 90 | 1387 | 1502 |
| 95 | 1657 | 1691 |
| 100 | 1801 | 1867 |
| 105 | 1951 | 2080 |
| 110 | 2107 | 2272 |
| 115 | 2437 | 2503 |
| 120 | 2611 | 2711 |
| 125 | 2791 | 2966 |
| 130 | 2977 | 3188 |
| 135 | 3367 | 3471 |
| 140 | 3571 | 3707 |
| 145 | 3781 | 4012 |
| 150 | 3997 | 4260 |
| 155 | 4447 | 4591 |
| 160 | 4681 | 4863 |
| 165 | 4921 | 5212 |
| 170 | 5167 | 5506 |
| 175 | 5677 | 5875 |
| 180 | 5941 | 6185 |
| 185 | 6211 | 6576 |
| 190 | 6487 | 6898 |
| 195 | 7057 | 7307 |
| 200 | 7351 | 7651 |
| 205 | 7651 | 8080 |
| 210 | 7957 | 8442 |
| 215 | 8587 | 8893 |
| 220 | 8911 | 9281 |
| 225 | 9241 | 9750 |
| 230 | 9577 | 10156 |
| 235 | 10267 | 10645 |
| 240 | 10621 | 11069 |
| 245 | 10981 | 11588 |
| 250 | 11347 | 12022 |

TABLE 2
Outer diameter (OD) of microfiber vs. the no. of pixels for two
arrangements with the following criteria of a Core Diameter
(CD): 3 micron (Color image or multi mode cores) and
edge-edge spacing between cores: 1 micron

| | Circular Arrangement | Rectangular Arrangement |
|---|---|---|
| OD | No. of Pixels | No. of Pixels |
| 80 | 271 | 289 |
| 85 | 331 | 339 |
| 90 | 331 | 378 |
| 95 | 397 | 417 |
| 100 | 397 | 462 |
| 105 | 469 | 516 |
| 110 | 547 | 561 |
| 115 | 547 | 618 |
| 120 | 631 | 667 |
| 125 | 721 | 739 |
| 130 | 721 | 798 |
| 135 | 817 | 853 |
| 140 | 817 | 914 |
| 145 | 919 | 998 |
| 150 | 1027 | 1065 |
| 155 | 1027 | 1132 |
| 160 | 1141 | 1203 |
| 165 | 1261 | 1303 |
| 170 | 1261 | 1376 |
| 175 | 1387 | 1447 |
| 180 | 1387 | 1532 |
| 185 | 1519 | 1644 |
| 190 | 1657 | 1725 |
| 195 | 1657 | 1810 |
| 200 | 1801 | 1897 |
| 205 | 1951 | 2021 |
| 210 | 1951 | 2118 |
| 215 | 2107 | 2203 |
| 220 | 2107 | 2304 |
| 225 | 2269 | 2440 |
| 230 | 2437 | 2545 |
| 235 | 2437 | 2642 |
| 240 | 2611 | 2737 |
| 245 | 2791 | 2893 |
| 250 | 2791 | 3006 |

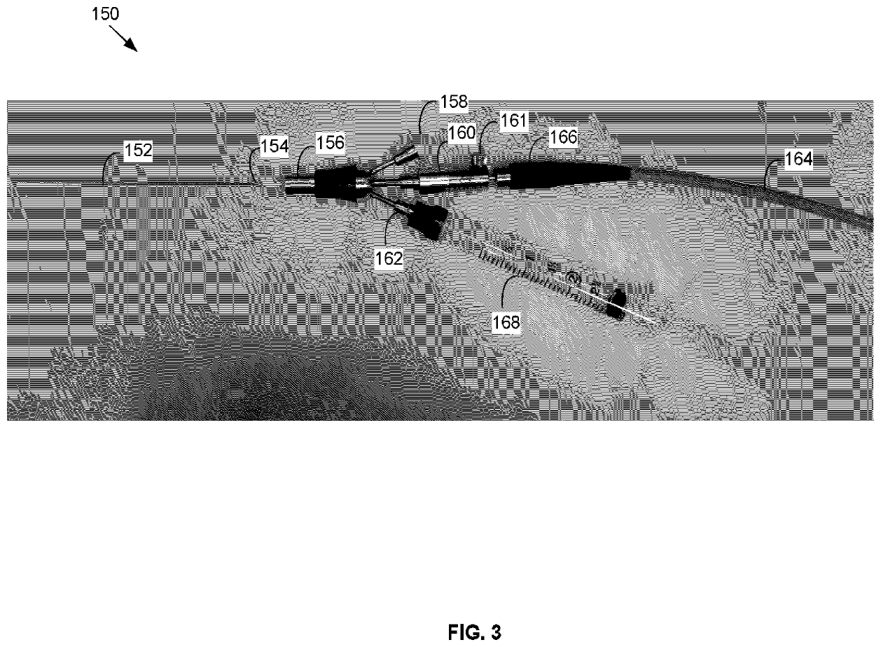

Referring now to FIG. 3, shown therein is a view of another example embodiment of an ultrafine needle endoscope 150 with additional attachments for providing increased functionality in accordance with the teachings herein. The endoscope 150 includes an ultrafine needle 152, a port 156, an optical cable 164 with a distal end 166, and a syringe 168. The port 156 has a connector 154, a first channel 160, a second channel 158 and a third channel 162. In this example embodiment, the ultrafine needle 152 is considered to be a polyshaft needle since it has a hollow shaft that may receive the fiber probe and still have empty space that can be used for various functions including at least one of imaging, suction and irrigation.

The connector 154 of the port 156 is adapted for removable connection to the ultrafine needle 152. The first channel 160 is adapted for coupling to the distal end 166 of the optical cable 164 for receiving excitation signals from the optical cable 164 and sending the excitation signals to the ultrafine needle 152 to the portion of the object being imaged. The first channel 160 is also adapted for receiving reflected light signals from the portion of the object being illuminated and sending the reflected light signals to the optical cable 164. The distal end 166 of the optical cable 164 is held in place using a set screw 161.

The second channel 158 of the port 156 is adapted to be coupled to a suction device (not shown) for collecting biopsy tissues and cells during use from the portion of the object being imaged.

The third channel 162 of the port 156 is adapted to be coupled to a rinsing device, such as the syringe 168, to provide a rinsing solution during use to the portion of the object being imaged.

In another aspect, in accordance with the teachings herein, there is provided a method for generating at least one image type of a portion of an object using an endoscope apparatus as defined in accordance with any of the embodiments described herein. An image type means a type of image that can be generated using the endoscope apparatus. For example, an image type can be a white light color image, an autofluorescence image or a Raman image.

The method for generating at least one image of a portion of an object using the endoscope 150, for example, may comprise: inserting a wire (not shown) into the ultrafine needle 152; inserting the wire and the ultrafine needle 152 into the portion of the object; removing the wire from the ultrafine needle 152; inserting a fiber probe into the ultrafine needle 152; attaching the optical cable 164 to the ultrafine needle 152 via the port 156; coupling a broadband light source 44 to the endoscope 150; generating at least one excitation light signal using the broadband light source 44 and transmitting the at least one excitation light signal to the portion of the object; receiving at least one reflected light signal from the portion of object; and generating at least one of a white light image, an autofluorescence image and a Raman spectral image from the at least one reflected light signal.

It should be noted that in some cases, the method involves creating an interstitial channel for inserting the wire and the first end of the ultrafine needle 152 into the object. To create the interstitial channel a regular trocar may be used. In some cases, a custom-made trocar may be used for acquiring biopsy samples. The trocar may be cut short to preserve the shape of its tip.

The object may be an ex vivo tissue or in vivo tissue.

The method may be used to generate images of various types of physiological tissue or organs such as, but not limited to, at least one of a thyroid image, a prostate image, and an ascites image, for example.

The method may further include coupling the endoscope to a rinsing device (e.g. the syringe 168) and providing a rinsing solution, such as saline, to portion of the object prior to performing imaging. The biggest obstacle in interstitial interrogation is the bleeding that obscures images which may be generated such as an autofluorescence image, for example. Accordingly, the rinsing device (also called an irrigation device) can be used to inject saline into the interstitial channel. The saline will then flush out the blood and which will allow clear images to then be taken after the cleansing is performed.

The method may further include coupling the endoscope to a suction device (not shown) and obtaining a biopsy sample from the portion of the object being imaged. For example, the method may further include obtaining various types of biopsies such as, but not limited to, at least one of a thyroid biopsy, a prostate biopsy, and an ascites biopsy, for example. In some cases, core needle biopsies may be obtained by using a magnum gun.

In another aspect, in accordance with the teachings herein, there is provided a use of an endoscope as defined in any of the embodiments described herein where the endoscope may be used to facilitate biopsies anywhere in the body such as, but not limited to, a thyroid biopsy, a prostate biopsy, or an ascites biopsy, for example.

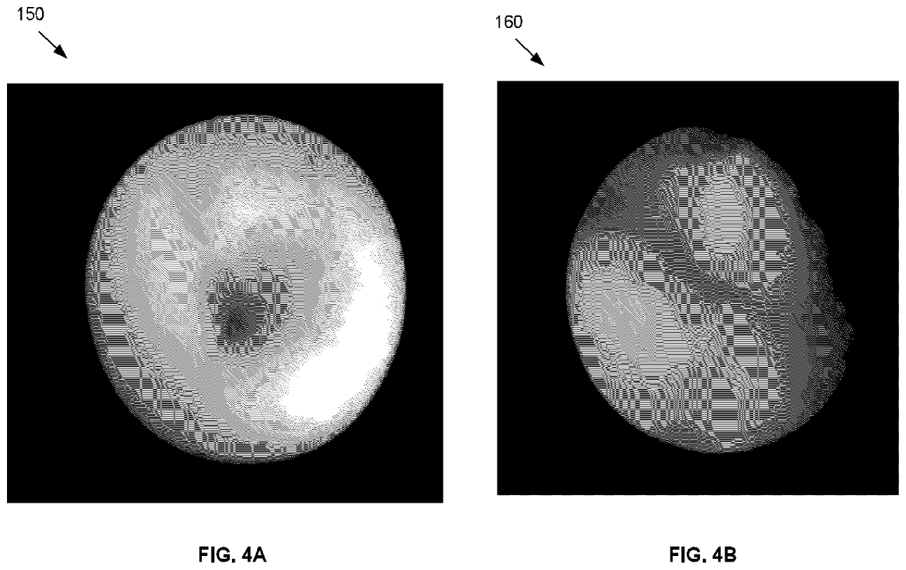

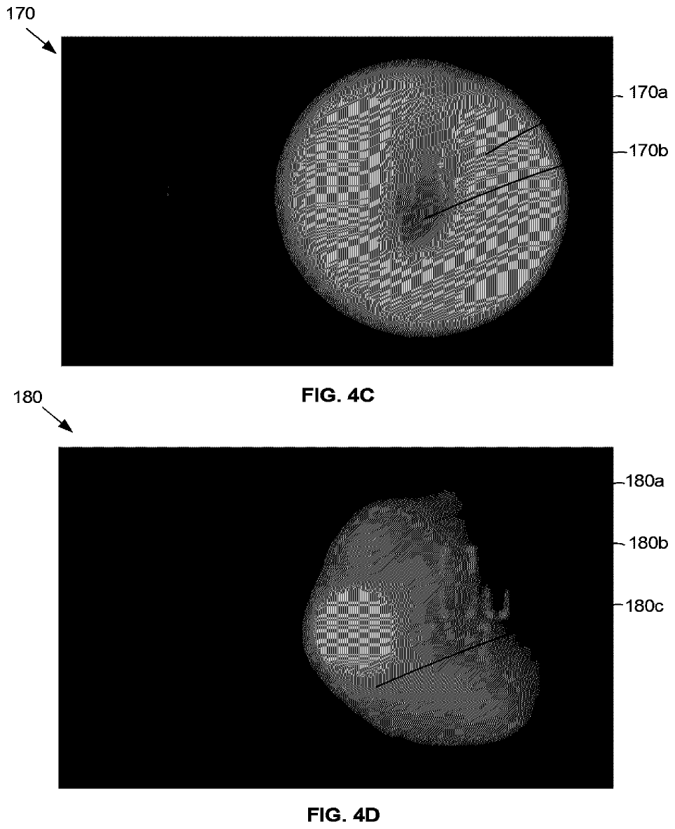

Referring now to FIGS. 4A-4B, shown therein are white light color images 150 and 160, respectively, that have been obtained using a prototype endoscope apparatus having an ultrafine needle endoscope with an outer diameter for the image guide of about 550 microns and being able to generate images with a resolution of about 6,000 pixels. The image 150 in FIG. 4A is of normal breast tissue. The image 160 in FIG. 4B is of cancerous breast tissue (3.5 cm invasive ductal carcinoma AJCC T2N1M0 (where AJCC is the American Joint Committee on Cancer, and T2N1M0 is a staging system where T refers to the size of the primary tumor, N is involvement of lymph nodes and M refers to if distant metastasis)).

It should be noted that as shown in Tables 1 to 2, the fiber bundle can vary from 80 to 120 microns in OD for black and white imaging (i.e. using single mode cores), the fiber bundle can have a configuration of optical fibers to provide from about 1,000 to 3,000 pixels in resolution while for color imaging (i.e. using multi-mode cores), the configuration of optical fibers in the fiber bundle can provide a resolution from about 200 to 700 pixels.

Referring now to FIGS. 4C-4D, shown therein are autofluorescence images 170 and 180, respectively, that have been obtained using the same prototype ultrafine needle endoscope on the same tissue region used to obtain the images shown in FIGS. 4A-4B. In this example embodiment of the endoscope apparatus, the optical assembly of the endoscope apparatus has emitted both blue and red light in the excitation signal. Initially, the blue light can be absorbed in the tissue and the tissue can then emit green light through fluorescence or diffuse reflected light, while the red light is diffusely reflected back though the optical assembly of the endoscope apparatus. After a short time, the blue light can be filtered out of the excitation signal. The accumulation of emitted light generates green autofluorescence images with red portions 180a, 180b, 180c for cancerous regions (as shown in FIG. 4D) or just different shades of green 170a, 107b for normal tissue (as shown in FIG. 4C).

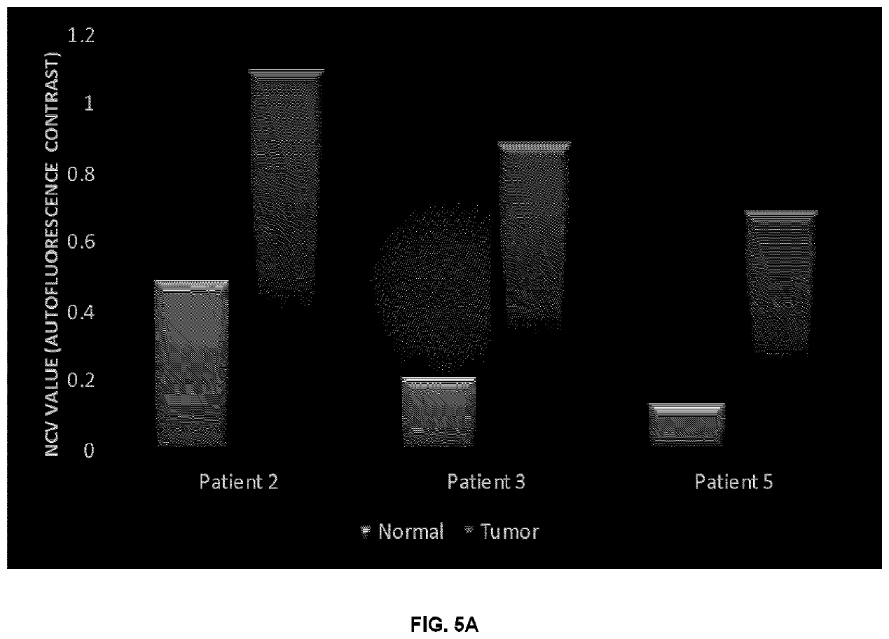

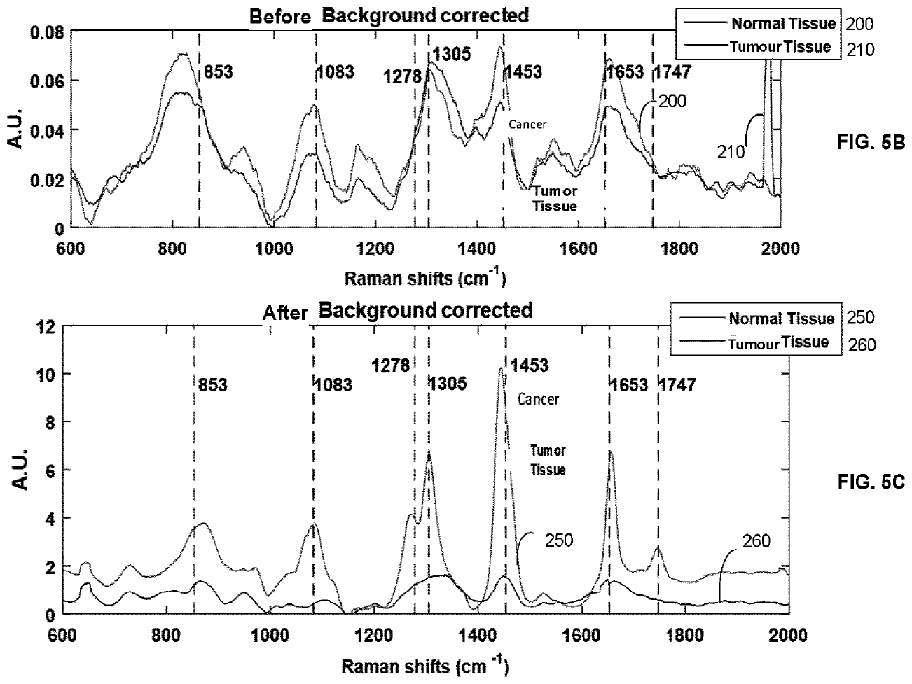

Referring now to FIG. 5A, shown therein are results for autofluorescence images obtained from ex vivo breast tissue samples obtained from several patients using a prototype ultrafine needle endoscope having a 550 micron outer diameter that provides 6,000 pixel resolution. The results are also shown in Table 3. The results show that the NCV values (indicating autofluorescence contrast) results are elevated in patients when autofluorescence images are generated of regions of their breast tissue that have a tumor compared to regions of their breast tissue that are normal. The NCV values for the normal tissue is the first bar for each patient while the NCV values for the tumor tissue in the second bar for each patient in FIG. 5A. The results indicate that autofluorescence diagnostics with good results can be obtained with this example endoscope.

TABLE 3
NCV Values (Autofluorescence Contrast)

| | Normal | Tumor |
|---|---|---|
| Patient 2 | 0.49 ± 0.17 | 1.10 ± 0.17 |
| Patient 3 | 0.21 ± 0.16 | 0.89 ± 0.45 |
| Patient 5 | 0.13 ± 0.05 | 0.69 ± 0.25 |

Referring now to FIGS. 5B and 5C, shown therein are Raman spectroscopy data. FIG. 5B shows results for Raman spectroscopy signals 200 and 210 obtained from ex-vivo breast tissue from a first patient using a prototype ultrafine needle endoscope having a 550 micron outer diameter before background correction. FIG. 5C shows results for Raman spectroscopy signals 250 and 260 obtained from ex-vivo breast tissue from a second patient using a prototype ultrafine needle endoscope having a 550 micron outer diameter after background correction where the Autofluorescence component of the background Raman signal is reduced.

The embodiments of the present disclosure described above are intended to be examples only and it is not intended that the applicant's teachings be limited to such embodiments. The present disclosure may be embodied in other specific forms. Alterations, modifications, and variations to the disclosure may be made without departing from the intended scope of the present disclosure. While the systems, devices, and processes disclosed and shown herein may comprise a specific number of elements/components, the systems, devices, and assemblies may be modified to include additional or fewer of such elements/components. For example, while any of the elements/components disclosed may be referenced as being singular, the embodiments disclosed herein may be modified to include a plurality of such elements/components. Selected features from one or more of the example embodiments described herein in accordance with the teachings herein may be combined to create alternative embodiments that are not explicitly describe. All values and sub-ranges within disclosed ranges are also disclosed. The subject matter described herein intends to cover and embrace all suitable changes in technology.

### REFERENCES

1. Gotzsche P C, Nielsen M., Screening for breast cancer with mammography. Cochrane Database Syst Rev 2006; (4): CD001877.

### 2. G B Taksler et al., Implications of False-Positive Results for Future Cancer Screenings, Cancer 2018:124 (11), 2390-2398.

3. Shen Y et al., The Impact of False Positive Breast Cancer Screening Mammograms on Screening Retention: A Retrospective Population Cohort Study in Alberta, Canada. Can J Public Health 2018:108 (5-6), e539-e545.

### 4. H Long et al., How Do Women Experience a False-Positive Test Result From Breast Screening? A Systematic Review and Thematic Synthesis of Qualitative Studies, Br J Cancer 2019:121 (4), 351-358.

### 5. Stiggelbout A, Copp T, Jacklyn G, Jansen J, Liefers G J, McCaffery K, Hersch J., Women's Acceptance of Overdetection in Breast Cancer Screening: Can We Assess Harm-Benefit Tradeoffs? Med Decis Making. 2020; 40 (1):42-51.

6. Mathioudakis A G et al., Systematic Review on Women's Values and Preferences Concerning Breast Cancer Screening and Diagnostic Services. Psychooncology 2019:28 (5), 939-947.
7. Hendrick R E and Alam M, Geisler A, Sadhwani D, et al., Effect of Needle Size on Pain Perception in Patients Treated With Botulinum Toxin Type A Injections: A Randomized Clinical Trial. JAMA Dermatol. 2015; 151 (11):1194-1199.
8. Hemmer J M, Kelder J C, van Heesewijk H P M, Stereotactic Large-Core Needle Breast Biopsy: Analysis of Pain and Discomfort Related to the Biopsy Procedure. Eur Radiol, 2008; 18 (2):351-4.

### 9. Douplik A., Leong W. L., Easson A. M., Done S., Netchev G., Wilson B. C., “A Feasibility Study of Autofluorescence Mammary Ductoscopy”, Journal of Biomedical Optics 2009; 14 (4), 044036 1-6.

10. Tissue Optics, light scattering methods and instruments for medical diagnostics, SPIE third edition Valery V Tuchin published: 2015, https://doi.org/10.1117/3.1003040.
11. Tu Q, Chang C. Diagnostic applications of Raman spectroscopy. Nanomedicine 2012; 8 (5):545-558.
12. Syringe Needle Gauge Chart | Sigma-Aldrich https://www.sigmaaldrich.com/chemistry/stockroom-reagents/learning-center/technicallibrary/needle-gauge-chart.html.

### 13. Lin J G, Chou P C, Chu H Y. An Exploration of the Needling Depth in Acupuncture: The Safe Needling Depth and the Needling Depth of Clinical Efficacy, Evid Based Complement Alternat Med. 2013; 2013:740508.

14. https://www.mayoclinic.org/tests-procedures/acupuncture/about/pac-20392763.
15. Praestmark K A, Jensen M L, Madsen N B, et al, Pen needle design influences ease of insertion, pain, and skin trauma in subjects with type 2 diabetes, BMJ Open Diabetes Research and Care 2016; 4:e000266.

1. An endoscope apparatus for obtaining light signals for generating at least one image of a portion of an object using at least one imaging modality, wherein the apparatus comprises:
an ultrafine needle having a body with first and second ends, the first end being adapted for insertion into the object;
a fiber probe that is slidably disposed in the ultrafine needle;
an optical cable having a body with first and second ends, the body having an outer cladding that surrounds a plurality of illumination optical fibers and a plurality of collection optical fibers, the first end of the optical cable forming the fibre probe;
a port having first and second ends, the first end of the port being coupled to the second end of the optical cable and the second end of the port being connected to at least one light source and at least one sensor where the at least one light source is adapted to provide at least one excitation light signal to the portion of the object to be imaged during use and the at least one sensor is adapted to receive reflected light signals from the portion of the object when it is illuminated during use for generating the at least one image.

2. An endoscope for obtaining light signals for generating multiple images of a portion of an object using several imaging modalities, wherein the endoscope comprises:
an ultrafine needle having a body with first and second ends, the first end being adapted for insertion into the object;
a fiber probe that is slidably disposed in the ultrafine needle, the fiber probe including a plurality of optical fibers or cores that act as illumination optical fibers and collection optical fibers;
an optical assembly that is coupled to the fiber probe, the optical assembly including:
at least one transmission optical pathway that is adapted to optically couple at least one of the optical fibers or cores with at least one light source via at least one set of optical elements to provide at least one excitation light signal to the portion of the object to be imaged during use; and
at least one return optical pathway that is adapted to optically couple, via the at least one set of optical elements, at least one of the optical fibers or cores that receive reflected light signals from the portion of the object when it is illuminated during use with at least one sensor for generating at least one image via.

3. The endoscope of claim 2, wherein the optical assembly comprises:
at least two transmission optical pathways that are adapted to optically couple at least one of the optical fibers or cores with at least two light sources via at least two sets of optical elements to provide at least two excitation light signals to the portion of the object to be imaged during use; and
at least two return optical pathways that are adapted to optically couple, via at least two second sets of optical elements, at least one of the optical fibers or cores, that receive reflected light signals from the portion of the object when it is illuminated during use, with at least two sensors for generating at least two images,
wherein the at least two excitation light signals and the at least two sensors are adapted for generating two or more of a white light image, an Autofluorescence image and a Raman image.

4. The apparatus of claim 2, wherein the body of the ultrafine needle has an outer diameter of about 200 microns and/or an inner diameter of about 120 microns.

5. (canceled)

6. The apparatus of claim 2, wherein the length of the ultrafine needle is about at least 3 to 4.5 cm or longer.

7. The apparatus of claim 2, wherein the optical fibers or cores are adapted to provide a resolution from about 250 to 6,000 pixels.

8. The apparatus of claim 2, wherein the optical assembly comprises:
a first transmission optical pathway that is adapted for sending a broadband excitation light signal from a first light source that is a broadband light source via a first set of optical elements to the objective for transmission to the portion of the object being imaged; and
a first return optical pathway that is adapted for sending first reflected signals from the portion of the object being imaged in response to the broadband excitation light signal from the objective to a camera sensor for white light color imaging and to a spectral imaging sensor for spectral imaging.

9. The apparatus of claim 2, wherein the optical assembly comprises:
a second transmission optical pathway for sending a second excitation light signal from a second light source that provides a 785 nm excitation light signal via a second set of optical elements to the objective for transmission to the portion of the object being imaged; and
a second return optical pathway for sending second reflected signals from the portion of the object being imaged in response to the second excitation light signal from the objective to a light sensor for obtaining Raman images for wavelengths at about 785 nm.

10. The apparatus of claim 2, wherein the optical assembly comprises:
a third transmission optical pathway for sending a third excitation light signal from a third light source that provides a 532 nm excitation light signal via a third set of optical elements to the objective for transmission to the portion of the object being imaged; and
a third return optical pathway for sending third reflected signals from the portion of the object being imaged in response to the third excitation light signal from the objective to a third light sensor for obtaining Fluorescence images or Raman and Fluorescence images for wavelengths less than about 765 nm.

11. The apparatus of claim 2, wherein the optical assembly further comprises a notch filter for eliminating cross-talk between different optical pathways during use.

12. The apparatus of claim 2, wherein the apparatus includes a second channel that is adapted to be coupled to a suction device for collecting biopsy tissues and cells during use from the portion of the object being imaged.

13. The apparatus of claim 2, wherein the apparatus includes a third channel that is adapted to be coupled to a rinsing device to provide a rinsing solution during use to the portion of the object being imaged.

14. (canceled)

15. A method for generating at least one image of a portion of an object using an endoscope apparatus as defined in claim 3, wherein the method comprises:
inserting the ultrafine needle into the object;
coupling a broadband light source to the fiber probe;
generating at least one excitation light signal using at least one light source;
receiving at least one reflected light signal from the portion of the object; and
transmitting the at least one reflected light signal to at least one sensor for generating a white light image, an autofluorescence image and/or a Raman spectral image, wherein the ultrafine needle contains the fiber probe.

16. The method of claim 15, wherein inserting the ultrafine needle into the object comprises:
inserting a wire into the ultrafine needle;
inserting the wire and the ultrafine needle into the object;
removing the wire from the ultrafine needle; and
inserting the fiber probe into the needle.

17. The method of claim 16, wherein the method comprises creating an interstitial channel for inserting the wire and a first end of the ultrafine needle into the object.

18. The method of claim 17, wherein the object is ex vivo tissue or in vivo tissue.

19. The method of claim 15, wherein the method comprises generating a thyroid image, a prostate image, a breast image or an ascites image.

20. The method of claim 15, wherein the method further comprises coupling the endoscope apparatus to a rinsing device and providing a rinsing solution to the object prior to performing imaging.

21. The method of claim 15, wherein the method further comprises coupling the endoscope apparatus to a suction device and obtaining a biopsy sample from the object.

22. The method of claim 21, wherein the method further comprises obtaining a thyroid biopsy, a prostate biopsy, or an ascites biopsy.

23. (canceled)

24. (canceled)
